(19) United States
(12) Patent Application Publication
(10) Pub. No.: US 2012/0232563 A1
(43) Pub. Date: Sep. 13, 2012

(54) IMPLANT CATHETERS FOR PHYSIOLOGICAL PACING

(75) Inventors: Terrell M. Williams, Brooklyn Park, MN (US); Steven L. Waldhauser, White Bear Township, MN (US)

(73) Assignee: Medtronic, Inc., Minneapolis, MN (US)

(21) Appl. No.: 13/042,510

(22) Filed: Mar. 8, 2011

(51) Int. Cl. A61B 19/00

(52) U.S. Cl. 606/129

(57) ABSTRACT

A catheter for implanting a cardiac pacing electrode within a right atrial septum, to stimulate the His bundle, includes a deflectable shaft having a wall that includes an adjustable segment, a pre-formed segment and a substantially straight distal segment. The pre-formed segment extends distally from a pull wire anchoring member of the adjustable segment and out of a single plane in which the adjustable segment is deflectable; and the substantially straight distal segment extends distally, directly from the pre-formed segment, along a plane oriented at an angle with respect to that in which the adjustable segment is deflectable, and over a length between approximately seven and nine millimeters. An arc through which the pre-formed segment extends is preferably greater than approximately 80 degrees and less than approximately 130 degrees, and the angle of the plane of the distal segment is preferably between approximately 40 and 60 degrees.

### TECHNICAL FIELD

The present invention pertains to catheters for implanting cardiac pacing electrodes, and more particularly to those configured to implant such an electrode at a location to stimulate the His bundle for physiological pacing.

### BACKGROUND

One or more cardiac pacing electrodes, for example, mounted to a distal portion of an elongate implantable medical electrical lead, are typically implanted within one or both of the right atrium and right ventricle, depending upon the need of a particular cardiac patient. A cardiac pacing electrode implanted in the right atrium (RA) can provide pacing stimulation therapy that preserves both atrial-ventricular synchronization and normal ventricular activation patterns, via conduction of the pacing stimulation through at least a portion of the heart's intrinsic conduction system.

With reference to FIG. 1A, which is schematic diagram of a right side of a heart having an anterior-lateral wall peeled back, parts of the heart's intrinsic conduction system are shown as follows: a sinoatrial (SA) node 1, an atrioventricular (AV) node 2, a His bundle 3, a right bundle branch 4 and Purkinje fibers 5. In a normal or healthy conduction system, an electrical impulse starting at SA node 1 travels rapidly through the wall of the RA 10 and of the left atrium (not shown) and to AV node 2, where the impulse slows to create a delay before passing through His bundle 3, which branches, within an intraventricular septum 7, into a right bundle branch 4 and a left bundle branch (not shown). The impulse then travels through the right and left bundle branches toward the apex 16 of the right ventricle RV 6 and the apex of the left ventricle LV (not shown) where it spreads through Purkinje fibers 5. Thus, flow of electrical impulses through the normal and healthy intrinsic conduction system creates an orderly sequence of atrial and ventricular contraction and relaxation, which efficiently pumps blood through the heart. But, if a portion of the intrinsic conduction system becomes compromised, for example, by injury or congenital defect, efficient pumping of blood is also compromised, so that pacing stimulation therapy, for example, via the above-referenced cardiac pacing electrode(s), may be necessary.

Those skilled in the art are familiar with physiological pacing, which may be generally defined as stimulation of the intrinsic conduction system of a heart in order to preserve a natural conduction pattern of the heart. Commonly-assigned U.S. Pat. No. 7,729,782 describes embodiments of a delivery catheter configured for implanting a pacing electrode of an elongate medical electrical lead in close proximity to the His bundle 3 for physiological pacing. Although these previously disclosed embodiments were found useful, the continued investigation of implant techniques for hearts of various sizes has resulted in an improved catheter, embodiments of which are described herein.

### SUMMARY

Embodiments of an improved catheter for implanting a cardiac pacing electrode at a location to stimulate the His bundle from within the right atrium (RA), include a deflectable shaft, which is deflectable by activation of a pull wire. The deflectable shaft wall includes: an adjustable segment, which extends distally, directly from a substantially straight proximal segment to a pull wire anchoring member, and which is deflectable in a single plane; a pre-formed segment extending distally from the pull wire anchoring member through an arc, which is greater than approximately 80 degrees and less than approximately 130 degrees, and which extends out of the single plane in which the adjustable segment is deflectable; and a substantially straight distal segment, which extends distally, directly from the pre-formed segment to the distal terminal end of the shaft, and along a plane that is oriented at an angle with respect to the single plane in which the deflectable segment is deflectable. According to some preferred embodiments, the angle of the plane of along which the distal segment extends is between approximately 40 degrees and approximately 60 degrees, and the substantially straight distal segment, which includes the distal terminal end of the shaft, preferably has a length between approximately seven millimeters and approximately nine millimeters.

### BRIEF DESCRIPTION OF THE DRAWINGS

The following drawings are illustrative of particular embodiments of the present invention and therefore do not limit the scope of the invention. The drawings are not to scale (unless so stated) and are intended for use in conjunction with the explanations in the following detailed description. Embodiments of the present invention will hereinafter be described in conjunction with the appended drawings wherein like numerals denote like elements.

FIG. 1A is a schematic diagram of a right side of a heart having an anterior-lateral wall peeled back to illustrate parts of the intrinsic conduction system of the heart.

FIG. 1B is a schematic diagram showing an interior portion of a right atrium of the heart and an approach, via a catheter, for implanting a cardiac pacing electrode, according to some embodiments.

FIG. 1C is a schematic cross-section taken along one of three planes introduced in FIG. 1B.

FIG. 2 is a plan view of a distal portion of a catheter, according to some embodiments of the present invention.

FIG. 3A is a plan view of the catheter in an un-deflected state, according to some embodiments.

FIG. 3B is a schematic showing some detail of the construction of a deflectable catheter shaft of the catheter of FIG. 3A, according to some embodiments.

FIGS. 3C-D are a plan view and a corresponding end view of the pre-formed segment and a distal segment of the deflectable catheter shaft, according to some embodiments.

FIG. 4 is a plan view of a distal portion of an implantable medical electrical lead positioned within the distal segment of the catheter shaft, according to some embodiments.

FIGS. 5A-B are longitudinal section and corresponding end views of a distal terminal end of the catheter shaft that includes optional electrodes.

### DETAILED DESCRIPTION

The following detailed description is exemplary in nature and is not intended to limit the scope, applicability, or configuration of the invention in any way. Rather, the following description provides practical illustrations for implementing exemplary embodiments of the present invention. Examples of constructions, materials, dimensions and manufacturing processes are provided for selected elements, and all other elements employ that which is known to those skilled in the field of the invention. Those skilled in the art will recognize that some of the examples may have suitable alternatives.

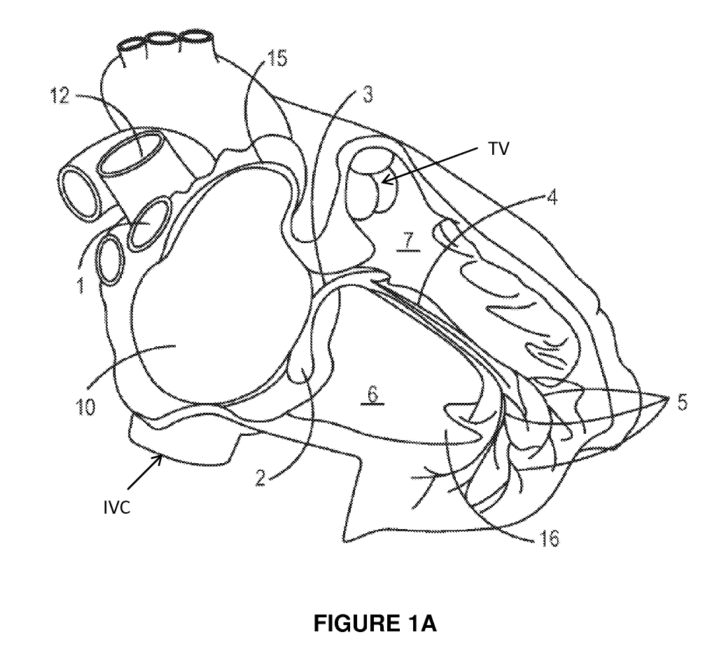

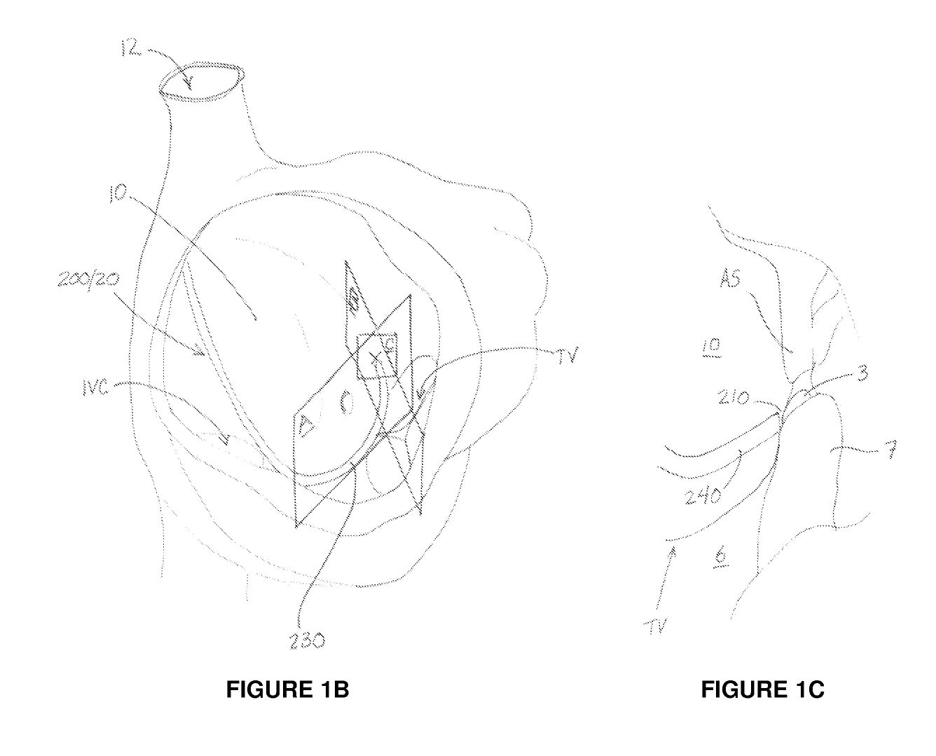

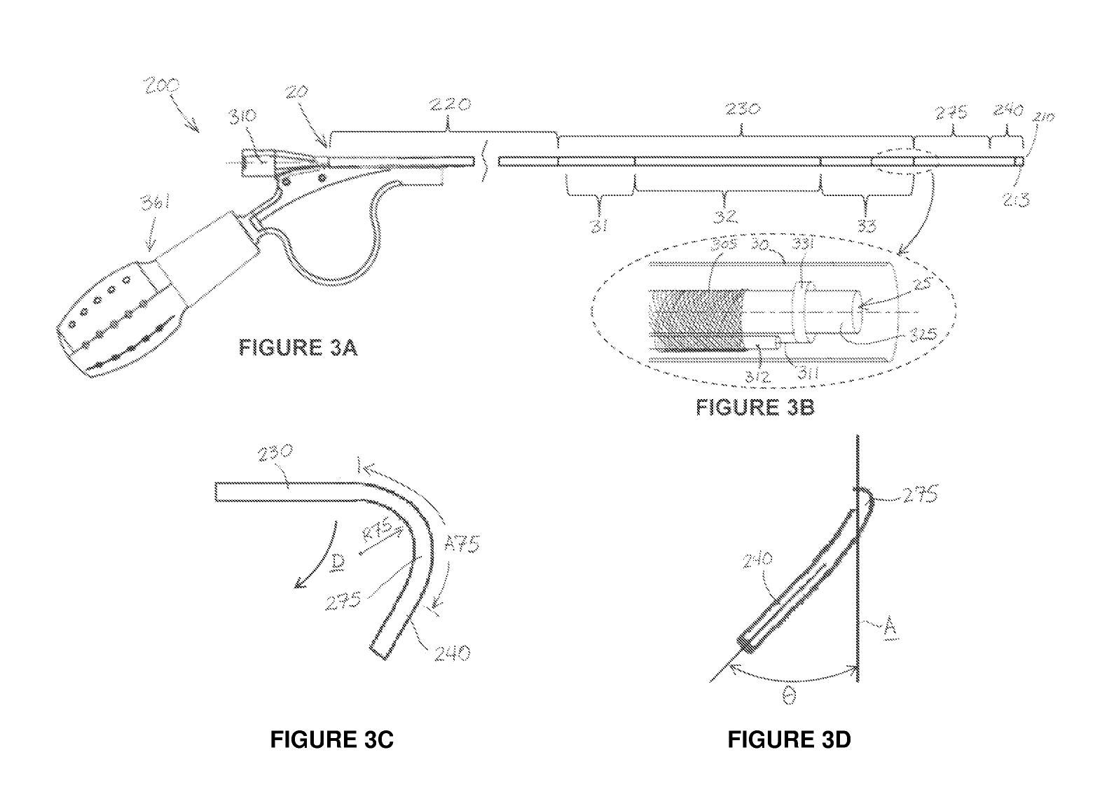

FIG. 1B is a schematic diagram showing an interior portion of RA 10. FIG. 1B illustrates a distal portion of a catheter 200 positioned in RA 10 so that a pacing electrode, for example, that terminates a distal portion of an elongate implantable medical electrical lead, may be implanted within a wall of RA 10, the atrial septum AS, at location X to stimulate His bundle 3 (FIG. 1A). According to embodiments of the present invention, catheter 200 includes a deflectable shaft 20 (FIG. 3A) having a wall that defines a longitudinally extending lumen 25 (FIG. 3B) through which a distal portion of an elongate implantable medical electrical lead can be advanced distally, from a proximal end of shaft 20, out through a distal opening of lumen 25, which is located at a distal terminal end 210 of shaft 20. FIG. 1B further illustrates three planes A, B, C superimposed on RA 10 in order to define relative orientations of some segments of the shaft wall of catheter 200, which orientations are suitable for directing the pacing electrode for implantation at location X. With reference to FIG. 1B, plane C corresponds to that portion of the atrial septum AS containing location X, plane B extends approximately perpendicular to plane C, and plane A extends at an angle between plane C and plane B. According to FIG. 1B, the distal portion of catheter 200 curves through an arc that lies on plane A and then extends distally from that curve out of plane A to distal terminal end 210, as shown in FIG. 1C. FIG. 1C is a schematic cross-section taken along plane B of FIG. 1B. FIG. 1C illustrates His bundle 3 within the atrial septum AS, at a crest of the ventricular septum 7 and in proximity to the tricuspid valve TV. FIG. 1C further illustrates distal terminal end 210 of catheter shaft 20, which extends along plane B, confronting the atrial septum AS so that the pacing electrode (not shown), once advanced out from lumen distal opening at distal terminal end 210, may be implanted therein to stimulate His bundle 3.

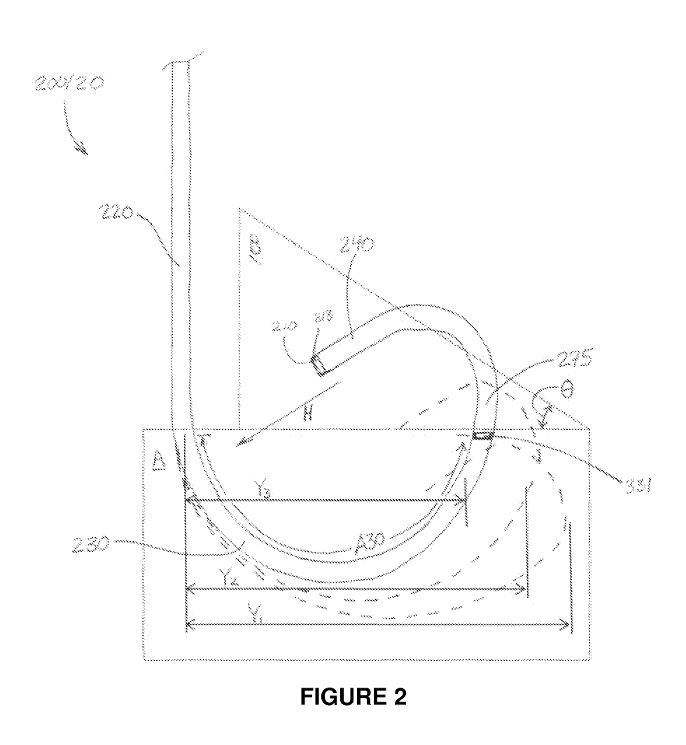

FIG. 2 is a plan view of the distal portion of catheter 200, according to some embodiments, with plane A and plane B superimposed thereon. FIG. 2 designates various segments of the deflectable shaft wall of catheter 200 as follows: a substantially straight proximal segment 220; an adjustable segment 230, which extends distally, directly from proximal segment 220, along plane A; a pre-formed segment 275, which extends distally from adjustable segment 230 through an arc that extends out of plane A; and a substantially straight distal segment 240, which extends distally from pre-formed segment 275, along plane B, and which includes distal terminal end 210. With reference to FIGS. 1B and 2, according to some embodiments, plane B is oriented at an angle θ, which is between approximately 40 degrees and approximately 60 degrees, preferably approximately 45 degrees, with respect to plane A. FIG. 2 further illustrates, with dashed lines, some alternate curvatures of adjustable segment 230 that are made possible by deflection of shaft 20, for example, via activation of a pull wire 311 (FIG. 3B), which is embedded within the wall of shaft 20. It should be noted that, according to preferred embodiments, adjustable segment 230 is deflectable in a single plane, which is designated plane A. According to some preferred embodiments, when adjustable segment 230 is fully deflected, adjustable segment 230 extends through an arc A30 of approximately 180 degrees that has a radius of curvature of approximately 40 millimeters; furthermore, it should be noted that a projection of substantially straight segment 240 onto plane A extends back toward adjustable segment 230, as indicated by arrow H.

FIG. 3A is a plan view of catheter 200 in an un-deflected state and prior to creating any curvature in pre-formed segment 275, according to some embodiments; and FIG. 3B is a schematic showing some detail of the construction of the wall of deflectable catheter shaft 20. FIG. 3A illustrates catheter 200 further including a hub 310, which surrounds a proximal opening into lumen 25 (FIG. 3B), and from which substantially straight proximal segment 220 of the deflectable shaft wall extends distally to adjustable segment 230, preferably over a length of between approximately 23 centimeters (9 inches) and approximately 32 centimeters (12.6 inches). It should be noted that, although proximal segment 220 is formed to be substantially straight in a resting state, proximal segment 220 is flexible enough to bend without kinking when catheter shaft 20 is inserted into a patient's venous system, for example, via a subclavian stick, and passed into the right heart, as illustrated in FIG. 1B. FIGS. 3A-B illustrate adjustable segment 230 extending distally, directly from proximal segment 220, to a pull wire anchoring member 331, preferably over a length between approximately 10 centimeters (4 inches) and approximately 19 centimeters (7.5 inches). With reference to FIGS. 3A-B, it may be appreciated that pull wire 311 extends within the shaft wall between a proximal attachment to a proximal control member 361 and a distal attachment to anchoring member 331. According to the illustrated embodiment, pull wire 311 is contained within a tubing member 312, which is embedded within the shaft wall between a lumen liner 325 and a reinforcing braid 305 that is fused together with an outer polymer layer 30 of the wall (shown peeled away in FIG. 3B). Braid 305 preferably extends along substantially straight proximal segment 220 and adjustable segment 230, from proximal control member 361 to anchoring member 331, while outer polymer layer 30 extends from hub 310 to distal terminal end 210, as does lumen liner 325 to provide a relatively lubricious interface for an elongate medical electrical lead passing within lumen 25. According to some exemplary embodiments: pull wire 311 is formed from a 304 stainless steel wire having a diameter of approximately 0.007 inch; tubing member 312 is formed by PTFE lined polyurethane tubing that includes reinforcing strands of 304 stainless steel wire embedded therein; and pull wire anchoring member 331 is formed from an 18K gold ring that surrounds lumen 25, is embedded in the shaft wall between liner 325 and outer layer 30, has a length of approximately 0.08 inch and a wall thickness of approximately 0.002 inch, and doubles as a radiopaque marker band. Control member 361 may be rotated to apply pull force to anchoring member 331, via tension in pull wire 311, in order to deflect adjustable section 230, for example as illustrated in FIG. 2. FIG. 3B shows a distal terminal end of braid 305 spaced apart, proximally, from anchoring member 331, however, according to some alternate embodiments, braid 305 is terminated at anchoring member 331. According to exemplary embodiments, reinforcing braid 305 is formed in an 8×8 pattern from round medium tensile 304 stainless steel wires, each having a diameter of approximately 0.002 inch.

FIG. 3A further illustrates adjustable segment 230 divided into a proximal transition section 31, a central section 32 and a distal transition section 33 that includes pull wire anchoring member 331. According to some preferred embodiments, the shaft wall is less stiff along central section 32 of adjustable segment 230 than along proximal and distal sections 31, 33, and a length of central section is between approximately 5 centimeters and approximately 10 centimeters. According to an exemplary embodiment, outer polymer layer 30 is divided into portions as follows: a first portion extending along proximal segment 220 that has a durometer of approximately 72 D; second and third portions extending along proximal and distal transition sections 31, 33 of adjustable segment 230 that each have a durometer of approximately 55 D; a fourth portion extending along central section 32 of adjustable segment 230 that has a durometer of approximately 40 D; and a fifth portion extending along pre-formed segment 275 that has a durometer of 35 D. (All indicated durometers are along the Shore D scale for hardness.) In some preferred embodiments, the fifth portion of outer layer 30 that extends along segment 275, for example, having the 35 D durometer, also extends along at least a portion of substantially straight distal segment 240. With further reference to FIG. 3A, substantially straight distal segment 240 is shown including an atraumatic distal tip 213 (coincident with distal terminal end 210), that may be formed from another, relatively soft, portion of polymer layer 30, for example, having a durometer of approximately 25 D. According to some preferred embodiments, the polymer forming tip 213 may include a radiopaque filler, such as tungsten carbide, in order to function as a marker band, either independently or in conjunction with pull wire anchoring member 331. If tip 213 and pull wire anchoring member 331 are both radiopaque, according to some preferred embodiments, the shaft wall along a combined length of pre-formed segment 275 and substantially straight distal segment 240, that extends between member 331 and tip 213, is significantly less radiopaque than either, so that pull wire anchoring member 331 and distal tip 213 may be fluoroscopically viewed as discrete entities, for example, as will be described below. The portions of polymer layer 30 may be formed from various grades of a thermoplastic elastomer such as PEBAX®, and deflectable shaft 20 constructed according to methods known to those skilled in the art.

FIGS. 3C-D are a plan view and a corresponding end view of pre-formed segment 275 and distal segment 240 of deflectable catheter shaft 20, according to some embodiments, once the curvature is formed in pre-formed segment 275. The curvature of pre-formed segment 275 may be fixed, for example, on a shaped mandrel by any suitable heat setting method, or by any other method known to those skilled in the art. However, it should be appreciated that the shaft wall along pre-formed segment 275 can flex in response to forces imposed when maneuvering catheter for positioning distal terminal end 210 at location X (FIG. 1C), so that the fixed pre-formed curvature of segment 275 may be temporarily deformed during the maneuvering. FIG. 3C illustrates pre-formed segment 275 extending from adjustable segment 230, which is deflectable in a single plane (plane A), as described above, per arrow D. FIG. 3C further illustrates segment 275 extending through an arc A75 that is greater than approximately 80 degrees and less than approximately 130 degrees, and, according to some preferred embodiments, a radius of curvature R75 of arc A75 is nine millimeters. As was indicated above, and with reference to the end view of FIG. 3D, pre-formed segment 274 curves out of the single plane (plane A) in which adjustable segment 230 is deflectable, such that substantially straight distal segment 240 extends along a plane (plane B) that is oriented at angle θ with respect to the single plane, wherein angle θ is between approximately 40 degrees and approximately 60 degrees, preferably approximately 45 degrees.

With reference back to FIGS. 1B-C and 2, further detail concerning the significance of each of the shaft wall segments 220, 230, 275 and 240 of catheter 200, in the context of implanting a cardiac pacing electrode, will now be described. Prior to passing a cardiac pacing electrode through catheter 200 for implantation, the operator inserts catheter shaft 20 into the patient's venous system, for example, via a subclavian stick. A dilator may be inserted within catheter shaft 20 to straighten out pre-formed segment 275 for initial insertion and, oftentimes, a guidewire will be positioned ahead of catheter 200 so that catheter shaft 20 (and dilator) can be directed over the guidewire, according to methods known in the art. The guidewire may be prepositioned to extend through the RA 10 and the tricuspid valve TV and into the RV 6, and then catheter shaft 20 and dilator advanced over the guidewire so that distal terminal end 210 of shaft 20 is located just below tricuspid valve TV within RV 6, at which point the operator removes the dilator and guidewire from lumen 25 of shaft 20. The operator may then begin to deflect adjustable segment 230 while pulling back catheter shaft 20 so that distal terminal end 210 passes back through tricuspid valve TV and into RA 10. The above-described curvature of pre-formed segment 275 in conjunction with the deflection of adjustable segment 230 orients distal segment 240 generally toward the RV septum 7 and the atrial septum AS, and the tactile feedback of distal segment 240 passing back through tricuspid valve TV and coming into contact with the crest of the RV septum 7 within the RA 10 helps the operator to maneuver distal terminal end 210 to location X in proximity with His bundle 3 (FIGS. 1B-C). Furthermore, according to those preferred embodiments in which tip 213 and pull wire anchoring member 331 are radiopaque, as was described above, in conjunction with FIGS. 3A-B, fluoroscopic visualization of tip 213 and anchoring member 331 can provide an indication of the proper orientation of distal terminal end 210 toward location X. For example, when viewed in a right anterior oblique projection (RAO), the proper orientation of end 210 may be indicated by the appearance/visualization of anchoring member 331 as a generally rectangular shape and of tip 213 as a generally circular shape, and vice versa, when viewed in a left anterior oblique projection (LAO). In some cases, an electrophysiology (EP) mapping catheter (not shown), which includes an array of electrode pairs pre-positioned within the RA 10 (advanced from a femoral insertion site up through the inferior vena cava IVC), may be used to further assist the operator in positioning distal terminal end 210 of catheter 200 at location X, for example, via electrical mapping to find and record the largest His bundle potential while maneuvering the mapping catheter by means of fluoroscopic visualization, preferably, in the RAO projection, according to methods known in the art.

Depending upon the size of the patient's heart, for example, ranging from a relatively small atrium of a female patient to a relatively large and dilated atrium of a male patient, the operator can continue to deflect adjustable segment 230 to a particular radius of curvature that will reach across the RA 10, from a position on a floor of RA 6, in proximity to the entrance of the inferior vena cava IVC, to the atrial septum AS (FIG. 1B), in order to position distal terminal end 210 against the atrial septum AS in proximity to location X. With reference to FIG. 2, the adjustable ‘reach’ of adjustable segment 230 is illustrated by some exemplary distances Y₁, Y₂ and Y₃, which are just a few of the many for segment 230. Once the distal opening of lumen 25, at distal terminal end 210, faces generally toward location X, a pacing electrode, for example, terminating a distal end of an elongate medical electrical lead, may be advanced out from the distal opening and implanted at location X to stimulate His bundle 3 for physiological pacing therapy.

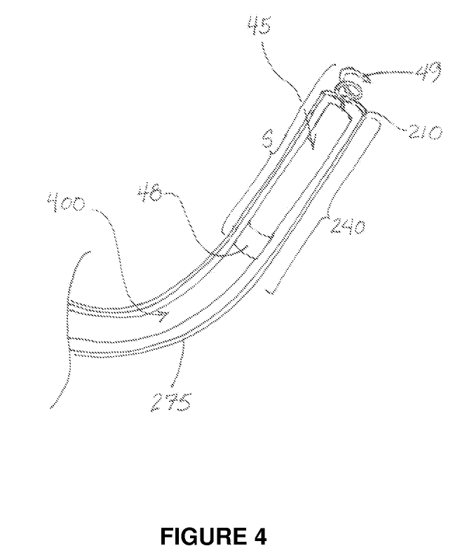

FIG. 4 is a plan view of a distal portion of an implantable medical electrical lead 400 positioned within substantially straight distal segment 240 of catheter shaft 20. FIG. 4 illustrates a pair of pace sense electrodes including a helical tip electrode 49 and a ring electrode 48, both of which are mounted to a distal segment 45 of lead 400. Segment 45 separates and isolates electrodes 48, 49 from one another and thereby establishes a spacing S between the electrodes for an effective sensing vector. Segment 45 may be relatively rigid compared with a length of lead 400 that extends proximally from electrode 48, for example, to provide some structural integrity between electrodes 48, 49 and/or to keep the sensing vector relatively constant between electrodes 48, 49 during implant. Those skilled in the art will appreciate that helical tip electrode 49 is designed for ‘screw-in’ fixation at an implant site, such as location X, by rotation of lead 400 from a proximal end thereof. According to some preferred embodiments, catheter shaft 20 has a lumen diameter, within pre-formed segment 275, in combination with a flexibility of preformed segment 275, that allows relatively easy passage of the relatively rigid segment 45 therethrough, in order to position tip electrode 49 for implant, as illustrated in FIG. 4. With reference back to FIGS. 3A-B, in order to accommodate a lead, such as lead 400, that has an outer diameter in the range from approximately 0.05 inch to approximately 0.06 inch along the relatively rigid segment 45, lumen liner 325 of an exemplary embodiment of catheter 200 has an inner diameter of approximately 0.08 inch, which corresponds to the diameter of lumen 25, and is formed from PTFE that has a wall thickness of approximately 0.0015 inch; and that portion of outer polymer layer 30 of the exemplary embodiment of catheter 200, which extends along pre-formed segment 275, has an outer diameter of approximately 0.110 inch, and is formed from PEBAX® that has a durometer of approximately 35 D. Of course, according to alternate embodiments, these exemplary dimensions can be scaled up or down to accommodate larger or smaller sizes of leads.

According to some preferred embodiments, as mentioned above, a length of substantially straight distal segment 240 is between approximately seven millimeters and approximately nine millimeters, which range of lengths accommodates a typical range of spacing S for electrodes 48, 49, yet is not so long to compromise a fit of the distal portion of catheter 200 reaching across the RA 10, as described above, in a relatively small heart, such as that of a female patient. Thus, when segment 45 of lead 400 is positioned as illustrated, such that tip electrode extends just outside of distal terminal end 210 of catheter for screw-in fixation at the implant site, pre-formed segment 275 does not impede rotation of lead 400 within catheter shaft 20, since the more flexible length of lead 400, that extends proximally from electrode 48 is positioned therein, due to the minimum specified length of substantially straight distal segment 240. If the length of substantially straight distal segment 240 were shorter than the typical spacing S, which is between approximately seven millimeters and approximately nine millimeters, segment 45 would be positioned within pre-formed segment 275, when electrode 49 is located at distal terminal end 210, and pre-formed segment 275 would ‘bind’ around the relatively rigid segment 45 to impede rotation of lead 400. Thus, as noted above, the length of segment 240 should be no less than approximately seven millimeters, but should not exceed approximately nine millimeters, in order that the distal portion of catheter 200 may fit within smaller sized hearts.

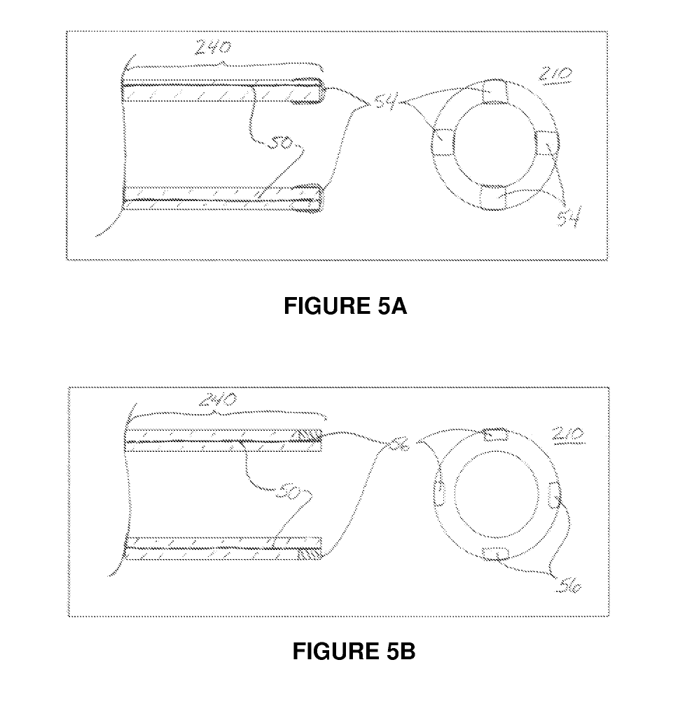

FIGS. 5A-B are longitudinal section and corresponding end views of distal segment 240 of catheter shaft 20 that includes optional electrodes 54, 56. FIG. 5A illustrates electrodes 54 mounted onto distal terminal end 210 so as to wrap around an end-facing surface thereof, and FIG. 5B illustrates electrodes 56 embedded into the shaft wall at distal terminal end 210. Electrodes may be formed from Platinum/Iridium alloy or any other suitable conductive and biocompatible material. With reference to the end views of FIGS. 5A-B, in conjunction with FIG. 1C, it may be appreciated that surfaces of electrodes 54, 56 are oriented to confront the atrial septum AS for electrical sensing/mapping of the location of His bundle 3. FIGS. 5A-B further illustrate conductors 50, each of which is coupled to a corresponding electrode 54, 56 and extends proximally through the shaft wall of distal segment 240; those skilled in the art will appreciate that conductors 50 extend into proximal segment 220 (FIG. 3A) to a proximal connector contact, for example, mounted in proximity to control member 361, and that conductors 50 may be integrated into shaft wall in a manner similar to that for pull wire 311 (FIG. 3B), according to methods known in the art. It should be noted that, although four of each electrode 54, 56 are illustrated in FIGS. 5A-B, two of each may alternately be incorporated; furthermore, alternate arrangements of optional electrodes 54, 56, about the perimeter of distal terminal end 210, may be employed.

In the foregoing detailed description, the invention has been described with reference to specific embodiments. However, it may be appreciated that various modifications and changes can be made without departing from the scope of the invention as set forth in the appended claims.

We claim:

1. A catheter for implanting a cardiac pacing electrode within a right atrial septum, at a location to stimulate the His bundle, the electrode being mounted to and terminating a distal portion of an elongate medical electrical lead, the catheter comprising a deflectable shaft having a wall that defines a longitudinally extending lumen through which the distal portion of the medical electrical lead can be advanced distally, from a proximal end of the shaft, out through a distal opening of the lumen, the distal opening being located at a distal terminal end of the shaft, the deflectable shaft being deflectable by activation of a pull wire, the pull wire extending from a proximal control member of the catheter to a pull wire anchoring member that is mounted within the wall of the deflectable shaft, and the deflectable shaft wall comprising:
a substantially straight proximal segment;
an adjustable segment extending distally, directly from the substantially straight proximal segment to the pull wire anchoring member, the adjustable segment being deflectable in a single plane;
a pre-formed segment extending distally from the pull wire anchoring member through an arc, the arc being greater than approximately 80 degrees and less than approximately 130 degrees and extending out of the single plane in which the adjustable segment is deflectable; and
a substantially straight distal segment extending distally, directly from the pre-formed segment to the distal terminal end of the shaft, along a plane that is oriented at an angle with respect to the single plane in which the deflectable segment is deflectable, the angle being between approximately 40 degrees and approximately 60 degrees, and the substantially straight distal segment including the distal terminal end of the shaft and having a length between approximately seven millimeters and approximately nine millimeters.

2. The catheter of claim 1, wherein:
the adjustable segment of the shaft wall includes a proximal transition section, a central section and a distal transition section, the proximal transition section extending between the substantially straight proximal segment of the shaft and the central section, the central section extending from the proximal transition section to the distal transition section, and the distal transition section extending from the central section to the pull wire anchoring member;
the proximal and distal transition sections have a first stiffness and the central transition section has a second stiffness, the first stiffness being greater than the second stiffness; and
a length of the central section is between approximately 5 centimeters and approximately 10 centimeters.

3. The catheter of claim 2, wherein the shaft wall further comprises:
a reinforcing braid extending along the substantially straight proximal segment and the adjustable segment, from the proximal control member of the catheter to a termination point within the distal transition section of the adjustable segment; and
the pre-formed segment and the substantially straight distal segment of the shaft wall are free of any reinforcing braid.

4. The catheter of claim 3, wherein the shaft wall further comprises an outer polymer layer that extends along the distal transition section of the adjustable segment and in which the reinforcing braid and the pull wire anchoring member are embedded, the outer polymer layer having a durometer of approximately 55 D.

5. The catheter of claim 3, wherein the shaft wall further comprises an outer polymer layer that extends along an entire length of the pre-formed segment and at least a portion of the substantially straight distal segment, the outer polymer layer having a durometer of approximately 35 D.

6. The catheter of claim 1, wherein, when the adjustable segment of the shaft wall is fully deflected:
the adjustable segment extends through an arc of approximately 180 degrees that has a radius of curvature of approximately 40 millimeters; and
a projection of the substantially straight segment of the shaft wall onto the single plane of the adjustable segment extends toward the adjustable segment.

7. The catheter of claim 1, wherein the angle of the plane along which substantially straight distal segment of the shaft wall extends is approximately 45 degrees.

8. The catheter of claim 1, wherein a radius of curvature of the arc through which the pre-formed segment of the shaft wall extends is approximately nine millimeters.

9. The catheter of claim 1, wherein the pull wire anchoring member comprises a radiopaque ring surrounding the lumen of the catheter.

10. The catheter of claim 9, wherein:
the substantially straight distal segment of the shaft wall includes a radiopaque distal tip coincident with the distal terminal end; and
the shaft wall along a combined length of the pre-formed segment and the substantially straight distal segment, which extends between the pull wire anchoring member and the radiopaque distal tip, is significantly less radiopaque than the pull wire anchoring member and the radiopaque distal tip.

11. A catheter for implanting a cardiac pacing electrode within a right atrial septum, at a location to stimulate the His bundle, the electrode being mounted to and terminating a distal portion of an elongate medical electrical lead, the catheter comprising a deflectable shaft having a wall that defines a longitudinally extending lumen through which the distal portion of the medical electrical lead can be advanced distally, from a proximal end of the shaft, out through a distal opening of the lumen, the distal opening being located at a distal terminal end of the shaft, the deflectable shaft being deflectable by activation of a pull wire, the pull wire extending from a proximal control member of the catheter to a pull wire anchoring member that is mounted within the wall of the deflectable shaft, and the deflectable shaft wall comprising:
a substantially straight proximal segment;
an adjustable segment extending distally, directly from the substantially straight proximal segment to the pull wire anchoring member, the adjustable segment being deflectable in a single plane and including a proximal transition section, a central section and a distal transition section, the proximal transition section extending between the substantially straight proximal segment of the shaft and the central section, the central section extending from the proximal transition section to the distal transition section, and the distal transition section extending from the central section to the pull wire anchoring member, the proximal and distal transition sections have a first stiffness and the central transition section has a second stiffness, the first stiffness being greater than the second stiffness, and a length of the central section is between approximately 5 centimeters and approximately 10 centimeters;
a pre-formed segment extending distally from the pull wire anchoring member through an arc, the arc being greater than approximately 80 degrees and less than approximately 130 degrees and extending out of the single plane in which the adjustable segment is deflectable, and a radius of curvature of the arc being approximately nine millimeters; and
a substantially straight distal segment extending distally, directly from the pre-formed segment to the distal terminal end of the shaft, along a plane that is oriented at an angle with respect to the single plane in which the deflectable segment is deflectable, the angle being approximately 45 degrees, and the substantially straight distal segment including the distal terminal end of the shaft and having a length between approximately seven millimeters and approximately nine millimeters;
wherein, when the adjustable segment of the shaft wall is fully deflected, the adjustable segment extends through an arc of approximately 180 degrees that has a radius of curvature of approximately 40 millimeters, and a projection of the substantially straight segment of the shaft wall onto the single plane of the adjustable segment extends toward the adjustable segment.

12. The catheter of claim 11, wherein the pull wire anchoring member comprises a radiopaque ring surrounding the lumen of the catheter.

13. The catheter of claim 12, wherein:
the substantially straight distal segment of the shaft wall includes a radiopaque distal tip coincident with the distal terminal end; and
the shaft wall along a combined length of the pre-formed segment and the substantially straight distal segment, which extends between the pull wire anchoring member and the radiopaque distal tip, is significantly less radiopaque than the pull wire anchoring member and the radiopaque distal tip.
